(19) United States
(12) Patent Application Publication
(10) Pub. No.: US 2024/0358256 A1
(43) Pub. Date: Oct. 31, 2024

(54) MULTI-DIMENSIONAL SIGNAL DETECTION WITH OPTICAL SENSORS

(71) Applicant: DEEPSIGHT TECHNOLOGY, INC., Santa Clara, CA (US)

(72) Inventors: Danhua ZHAO, San Jose, CA (US); Jiangang ZHU, University City, MO (US); Lan YANG, Clayton, MO (US); John Matthew CHONG, Ithaca, NY (US)

(21) Appl. No.: 18/685,985

(22) PCT Filed: Aug. 23, 2022
(86) PCT No.: PCT/US2022/041252

§ 371 Date: Feb. 23, 2024

Related U.S. Application Data
(60) Provisional application No. 63/236,610, filed on Aug. 24, 2021.

(51) Int. Cl. A61B 5/00; A61B 5/01

(52) U.S. Cl. CPC A61B 5/0059; A61B 5/01; A61B 5/6885; A61B 5/7228; A61B 5/7278; A61B 2560/0242

(57) ABSTRACT

Systems and methods for detecting multiple physical features is described herein. The method may include receiving a sensor signal from a single optical sensor proximate to a measurement region, determining a plurality of sensor responses from the sensor signal, and generating a plurality of measurement signals from the plurality of sensor responses. Each of the measurement signals may correspond to a different respective physical signal of the measurement region.

### CROSS-REFERENCE TO RELATED APPLICATIONS

This application claims the benefit of U.S. Provisional Application No. 62/236,610, filed Aug. 24, 2021, the content of which is hereby incorporated by reference in its entirety.

### TECHNICAL FIELD

Devices, systems, and methods herein relate to optical sensors.

### BACKGROUND

In many applications, it is desirable to detect multiple kinds of physical parameters. For example, in the field of medical technology, it may be advantageous to have medical devices with sensors that can sense multiple different physical parameters (e.g., simultaneously in real-time or near real-time). For example, ablation catheters for cardiovascular procedures may include a temperature sensor to measure a temperature of the treated tissues and a force sensor to measure a force applied to an arterial wall during heart ablation. It may be possible to incorporate multiple kinds of sensors together in a single device to monitor multiple different kinds of parameters. However, it may be more difficult to include additional sensors, because, for example, it may be more challenging to fit multiple sensors into a desired form factor of the device. Additionally or alternatively, the inclusion of more sensors may pose more difficulties in accommodating additional components (e.g., mechanical, electrical, power) and connections to enable proper functioning of each of the different sensors. Accordingly, it may be desirable to provide an optical sensor configured to sense a plurality of physical parameters.

### SUMMARY

Methods and system for multi-dimensional sensing with optical sensors are described herein. In some variations, a method for multi-dimensional sensing may comprise receiving a sensor signal from a single optical sensor proximate to a measurement region, determining a plurality of sensor responses from the sensor signal, and generating a plurality of measurement signals from the plurality of sensor responses. Each of the measurement signals may correspond to a different respective physical signal of the measurement region.

In some variations, a sensor response of the plurality of sensor responses may be selected from the group consisting of a mode shift, a baseline drift, a mode split, a mode broadening, and any combination thereof. In some variations, the sensor response may be a mode shift comprising change in one or more of resonant frequency, a change in depth, a change in shape, or any combination thereof. In some variations, the physical signals of the environment may be at least two of temperature, pressure, and an acoustic wave of the environment.

In some variations, generating the plurality of measurement signals may further comprise decoupling at least a portion of the physical signals. Decoupling at least a portion of the physical signals may comprise analyzing a first sensor response of the plurality of sensor responses for a first time period. A first measurement signal for the first time period may be generated based on the first sensor response. A second sensor response of the plurality of sensor responses may be analyzed for a second time period. A second measurement signal for the second time period may be generated based on the second sensor response. In some variations, the method may further comprise analyzing a third sensor response for a third time period and generating a third measurement signal for the third time period based on the third sensor response. In some variations, the first response may comprise a mode shift and the first measurement signal may correspond to temperature. The second sensor response may comprise a baseline shift and the second measurement signal may correspond to pressure.

In some variations, decoupling at least a portion of the physical signals may comprise selectively altering the environment based on a targeted physical signal. In some variations, decoupling at least a portion of the physical signals may comprise suppressing one or more of the physical signals different from the targeted physical signal. In some variations, suppressing one or more of the physical signals may comprise adjusting the environment such that the one or more physical signals different from the target physical signal is within a first sensitivity signal region of the optical sensor. In some variations, suppressing the one or more physical signal may comprise modifying at least one of a temperature, a pressure, and an acoustic property of the environment.

In some variations, decoupling at least a portion of the physical signals may comprise increasing a sensitivity of the optical sensor to the targeted physical signal. In some variations, increasing the sensitivity of the optical sensor may comprise adjusting an environment of the sensor such that the target physical signal is within a second sensitivity signal region of the optical sensor different from the first sensitivity signal region. For example, the second sensitivity signal region may be higher than the first sensitivity signal region. In some variations, increasing the sensitivity may comprise analyzing the first sensor response of the plurality of sensor responses for a first time period associated with the first sensitivity signal region and generating a first measurement signal corresponding to the target physical signal.

In some variations, generating the plurality of measurement signals may comprise associating a first sensor response of the plurality of sensor responses with a first physical signal, and associating a second sensor response of the plurality of sensor responses with a second physical signal. In some variations, generating the plurality of measurement signals may comprise applying a signal transformation function to the plurality of sensor responses. The signal transformation function may comprise a signal transformation matrix.

In some variations, the optical sensor may comprise a first optical sensor in an array of optical sensors. In some variations, the sensor signal may comprise a first sensor signal and the plurality of sensor responses may comprise a first plurality of sensor responses, the method may further comprise receiving a second sensor signal from a second optical sensor in the array of optical sensors, determining a second plurality of sensor responses from the second optical sensor, and generating a first measurement signal indicative of a first physical signal. The first measurement signal may be based on the first plurality of sensor responses for the first optical sensor, the second plurality of sensor responses for the second optical sensor, and sensitivities of the first and second optical sensors to the first physical signal and a second physical signal.

In some variations, the plurality of measurement signals may be generated at least in part based on a reference signal from a reference sensor. In some variations, the optical sensor may comprise an interference-based optical sensor. In some variations, the optical sensor may comprise an optical resonator or an optical interferometer. In some variations, the optical sensor may comprise a whispering gallery mode (WGM) resonator. In some variations, the optical sensor may comprise one or more of a microbubble optical resonator, a microsphere resonator, a micro-toroid resonator, a micro-ring resonator, and a micro-disk optical resonator.

Also described herein is a system for multi-dimensional sensing of a measurement region is described herein. The system may comprise an optical sensor, and a signal processor. The signal processor may be configured to receive a sensor signal from the optical sensor, determine a plurality of sensor responses from the sensor signal, and generate a plurality of measurement signals from the plurality of sensor responses. Each measurement signal may correspond to a different respective physical signal of the measurement region.

In some variations, a sensor response of the plurality of sensor responses may be selected from the group consisting of a mode shift, a baseline drift, a mode split, a mode broadening, and any combination thereof. In some variations, the sensor response may be a mode shift comprising one or more of a change in resonant frequency, a change in depth, a change in shape, or any combination thereof.

In some variations, the physical signals of an environment may comprise two or more of a temperature, a pressure, and an acoustic wave of the environment. In some variations, the optical sensor may comprise an interference-based optical sensor. In some variations, the optical sensor may comprise an optical resonator or an optical interferometer. In some variations, the optical sensor may comprise a whispering gallery mode (WGM) resonator. In some variations, the optical sensor may comprise one or more of a microbubble resonator, a microsphere resonator, a microtoroid resonator, a microring resonator, a microbottle resonator, a microcylinder, or a microdisk optical resonator. In some variations, the optical sensor may comprise a microring resonator comprising one or more of a cross-sectional shape of a circle, racetrack, and ellipse.

In some variations, the optical sensor may comprise a first optical sensor of an array of optical sensors, the system may further comprise the array of optical sensors. In some variations, the array of optical sensors may comprise a second optical sensor. The first optical sensor may have a higher sensitivity to a first physical signal than the second optical sensor and the second optical sensor may have a higher sensitivity to a second physical signal than the first optical sensor. The first and second physical signals may be different.

In some variations, an environment of the first optical sensor may be configured to enhance the first physical signal, or an environment of the second optical sensor may be configured to suppress the first physical signal, or both. In some variations, an environment of the first optical sensor may be configured to suppress the second physical signal, or an environment of the second optical sensor may be configured to enhance the second physical signal, or both. In some variations, the system may further comprise a reference sensor configured to provide a reference signal corresponding to one or more of the physical signals of the measurement region. In some variations, the reference sensor may comprise an optical sensor. In some variations, the reference sensor may comprise an non-optical sensor.

### BRIEF DESCRIPTION OF THE DRAWINGS

FIG. 1 depicts an exemplary variation of a whispering gallery mode (WGM) microsphere resonator.

FIG. 2 is a schematic block diagram of an exemplary variation of a system comprising an optical sensor configured to measure physical signals such as temperature and pressure.

FIG. 3 is a schematic block diagram of an exemplary variation of a system for sensing a plurality of physical signals.

FIG. 4 is a flowchart of an exemplary variation of a method for sensing a plurality of physical signals using a single optical sensor.

FIG. 5 is a schematic of an exemplary variation of an optical sensor for sensing a plurality of physical signals.

FIGS. 6A-6C depict exemplary optical sensor signals in response to nominal conditions, a pressure change, and a temperature change, respectively, in a measurement region proximate to the optical sensor.

FIG. 7A is an exemplary plot showing a sensor response curve with a sensitive region.

FIG. 7B is an exemplary plot showing a sensor response curve with an insensitive region.

FIG. 8 is a schematic block diagram of an exemplary variation of a system for performing multi-dimensional sensing by decoupling individual physical signals detected by a sensor.

FIG. 9 is a schematic block diagram of an exemplary variation of a system for performing multi-dimensional sensing by decoupling individual physical signals detected by an optical sensor by altering an environment of the optical sensor.

FIG. 10 illustrates a schematic block diagram of an exemplary variation of a system configured to alter the environment of a sensor to adjust sensitivity of the sensor.

FIG. 11 is a schematic of an exemplary variation of a system for performing multi-dimensional sensing by differentiating a plurality of physical signals detected by a sensor.

FIG. 12 is a schematic of an exemplary variation of a system for performing multi-dimensional sensing by combining a plurality of sensor responses of a sensor.

### DETAILED DESCRIPTION

Non-limiting examples of various aspects and variations of the invention are described herein and illustrated in the accompanying drawings.

Described herein are systems and methods for sensing (e.g., detecting, measuring, determining) a plurality of physical signals (e.g., temperature, pressure). For example, a single optical sensor may be configured to detect a plurality of physical signals which may enable, for example, accurate measurement of a plurality of physical signals substantially simultaneously using a single sensor. A physical signal may correspond to a physical property or characteristic associated with a state of a physical system.

Sensors configured to measure a plurality of physical signals can be useful for various applications. For instance, some medical devices such as cardiovascular treatments, cardiovascular monitoring and diagnosis, patient monitoring and diagnosis, surgical treatment, and the like may require detection of more than one physical signal. Similarly, Internet of Things (IoT) devices with applications in consumer (e.g., smart home), organizational (e.g., healthcare, transportation), and industrial (e.g., manufacturing) often require measurement of more than one physical signal.

Traditionally, devices and systems incorporate multiple different sensors to detect various physical signals, where each sensor is configured to detect a separate respective physical signal. For example, a device may incorporate a temperature sensor to measure temperature and a separate pressure sensor to measure pressure. Having separate sensors to detect different physical signals can, however, cause devices to be bulkier and/or pose design challenges since separate sensors require more physical volume for packaging, and may need separate electronics and connections. Having separate sensors may also make devices less reliable since there are additional components that may be susceptible to damage and failure.

Accordingly, it may be advantageous for a device or system to have a single sensor that can perform multi-dimensional sensing (e.g., measure a plurality of different physical signals substantially simultaneously in real-time or near real-time). Described herein are systems and methods for such multi-dimensional sensing. This may reduce one or more of a size, energy requirements, signal interference, and cost of a device and/or system.

### Optical Sensor Systems

An optical sensor system configured to measure a plurality of physical signals may generally include one or more optical sensors where an optical sensor (e.g., single sensor) may be used to detect multiple physical signals, such as temperature, pressure, acoustic waves, and the like by analyzing sensor responses such as a mode shift (e.g., change in frequency, depth, shape of response), a baseline drift, a mode split, and a mode broadening. The optical sensors described herein may advantageously have high sensitivity and broad bandwidth compared to conventional sensors.

In some variations, an optical sensor may include an interference-based optical sensor, such as an optical interferometer, an optical resonator, and the like. Examples of optical interferometers may include a Mach-Zehnder interferometer, a Michelson interferometer, a Fabry-Perot interferometer, a Sagnac interferometer, and the like. For example, a Mach-Zehnder interferometer may include two nearly identical optical paths (e.g., fibers, on-chip silicon waveguides). The two optical paths may comprise finely adjusted acoustic waves (e.g., by physical movement caused by the acoustic waves, tuning of refractive index caused by the acoustic waves) configured to distribute optical powers in an output(s) of the Mach-Zehnder interferometer for detecting a presence or a magnitude of the acoustic waves.

Generally, an optical resonator may include a closed loop transparent medium that allows a set of predetermined frequencies of light to continuously propagate within the closed loop, and to store optical energy of the predetermined frequencies of light in the closed loop. For example, an optical resonator may comprise a whispering gallery mode (WGM) resonator, where the WGM resonator may be configured to permit propagation of whispering gallery modes (WGMs) (e.g., waves) traveling a concave surface of the optical resonator corresponding to the predetermined frequencies circulating along a circumference of the resonator. Each mode of the WGM resonator may correspond to propagation of a frequency of light from the predetermined frequencies of light. The predetermined frequencies of light and a quality factor of the optical resonator may be based at least in part on a set of geometric parameters of the optical resonator, a refractive index of the transparent medium, and a set of refractive indices of an environment surrounding the optical resonator.

In some variations, a WGM resonator may include a substantially curved portion (e.g., a spherical portion, a toroid-shaped portion, a ring-shaped portion). The substantially curved portion may be supported (e.g., coupled, attached, integrated) by a stem portion. The shape of a WGM resonator (e.g., the shape of the substantially curved portion of the WGM resonator) can be any suitable shape. For example, the shape of the WGM resonator can be spherical (i.e., a solid sphere), bubble shaped (i.e., spherical shape with a cavity), cylindrical, elliptical, ring, disk, toroid, and the like. Some non-limiting examples of WGM resonators include microring resonators (e.g., circular microring resonators, non-circular microring resonators such as resonators having a shape of racetrack, ellipse), microbottle resonators, microbubble resonators, microsphere resonators, microcylinder resonators, microdisk resonators, microtoroid resonators, combinations thereof, and the like.

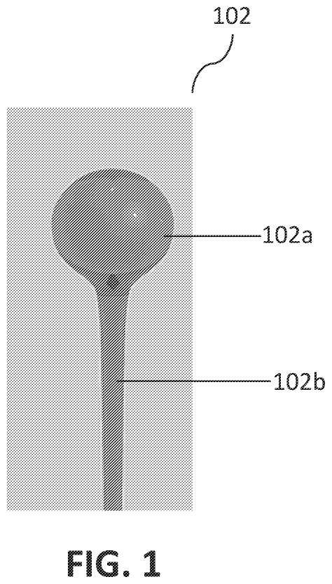

FIG. 1 is an exemplary variation of a WGM microsphere resonator 102. The WGM microsphere resonator 102 can include a substantially curved portion 102a (e.g., a spherical portion). The substantially curved portion 102a may be supported by a stem portion 102b. In some variations, the substantially curved portion 102a of the WGM microsphere resonator 102 may be coupled to or integral with an end of a stem portion 102b of the WGM microsphere resonator 102.

As discussed above, the WGM microsphere resonator 102 may be configured to trap (e.g., hold, capture, retain) a predetermined set of frequencies of light. The predetermined set of frequencies of light may be configured to circulate in the substantially curved portion 102a of the WGM microsphere resonator 102, thereby permitting propagation of whispering gallery modes along a surface of the WGM microsphere resonator 102 (e.g., along the circumference of the substantially curved portion 102a). In some variations, each set of WGMs propagated by the WGM microsphere resonator 102 may be confined to one or more planes within the WGM microsphere resonator 102.

Although the WGM microsphere resonator shown in FIG. 1 has a spherical shape, the substantially curved portion 102a can be any suitable shape. Generally, one or more of the performance characteristics of a WGM resonator 102 may depend on its shape. For example, in general a more spherical microsphere may be more effective in confining WGMs within the WGM resonator. Some suitable variations of the WGM microsphere resonator 102 may be elliptical (e.g., with some degree of eccentricity, such as between about 0 and about 0.9), and as described herein.

In order to sense light efficiently using an optical sensor, phase matching between incoming light and resonant light may be required. In some variations, an optical waveguide configured to provide phase matching may be used to couple light to an optical sensor. Optical waveguides may be configured to provide controllable and robust light capable of efficiently utilizing the sensing capabilities of an optical sensor. Accordingly, in some variations, sensor systems may include at least one optical waveguide and one or more optical sensors.

In some variations, at least a portion of the optical waveguide and optical sensor may be embedded into a polymer structure, thereby encapsulating the optical waveguide and the optical sensor. The polymer structure may protect the optical sensors and the optical waveguides from physical damage where optical sensors may be particularly fragile and susceptible to physical damage. In some variations, an effective refractive index of the polymer structure may be lower than an effective refractive index of the optical sensor. This may allow the packaged optical sensor to respond to a broad range of frequencies.

Further examples of optical systems (e.g., types of optical sensors, manufacturing and packaging of optical sensors) that may be used for multi-dimensional sensing are described in International Patent App. No. PCT/US2020/064094, International Patent App. No. PCT/US2021/022412, and International Patent App. No. PCT/US2021/039551, each of which is incorporated herein by reference.

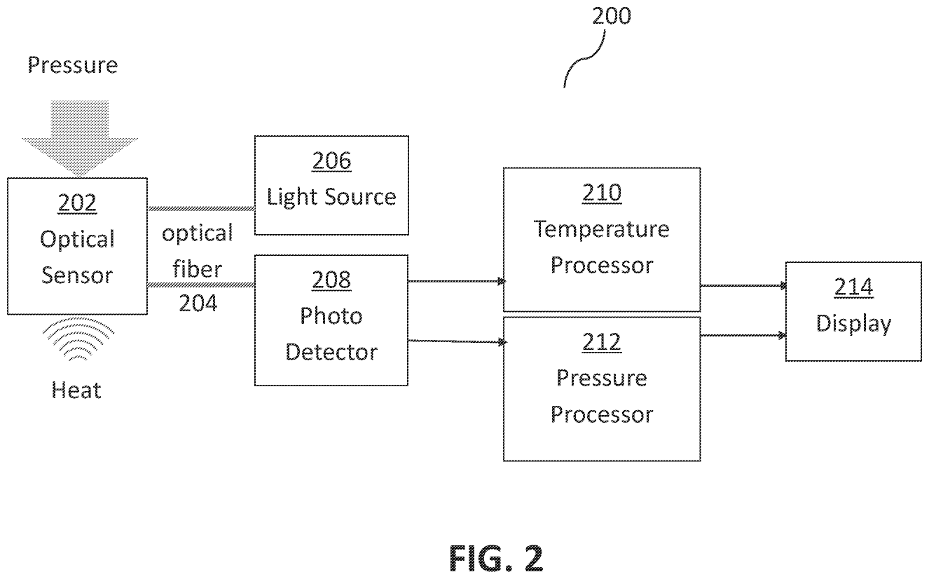

FIG. 2 is a schematic of an exemplary variation of a system 200 for sensing a plurality of physical signals. The system 200 may comprise an optical sensor 202, an optical waveguide 204, a light source 206, a photodetector 208, a temperature processor 210, a pressure processor 212, and an optional display 214. In some variations, the optical sensor 202 may be configured to measure a plurality of physical signals, such as temperature and pressure, within or proximate to a measurement region. As seen in FIG. 2, an optical sensor 202 (e.g., an optical resonator such as a WGM resonator) may be configured to be optically coupled to (e.g., in optical communication with) a light source 206 (e.g., a laser) and a photodetector 208 (e.g., optical detector, photoemission) via on or more optical waveguides 204 (e.g., an optical fiber, a slab waveguide). For example, the light source 206 may be configured to transmit light to the optical sensor 202 via the optical waveguide 204. The photodetector 208 may be configured to receive modulated light signals from the optical sensor 202 via the optical waveguide 204 and convert the received optical signals (e.g., sensor signal) from the optical sensor 202 into electrical signals. As described in further detail below, the photodetector 208 may be communicably coupled to one or more processors configured to determine a plurality of sensor responses (e.g., measurement data) from the electrical signals from the photodetector 208 (e.g., a temperature processor 210 for determining temperature data, a pressure processor 212 for determining pressure data). The processor(s) may be communicably coupled to a component for handling the data (e.g., input/output device) such as a display 214, memory device (not shown), and the like.

In some variations, the optical sensor 202 may comprise any suitable optical sensor such as those described above (e.g., a WGM resonator configured to propagate WGMs). In some variations, the optical sensor 202 may, for example, comprise a diameter of less than about 200 μm.

Although a single optical sensor 202 is shown in FIG. 2, it should be understood that in some variations, a system 200 may include an optical sensor array, where at least one of the optical sensors 202 may be configured to generate a sensor signal used to generate a plurality of measurement signals corresponding to a plurality of physical signals. Accordingly, while an array may include multiple optical sensors, each (or at least one) optical sensor in the array may function, independently and singularly from the other optical sensors in the array, as a multi-dimensional sensor capable of measuring a plurality of physical signals.

As described above, a light source 206 may be configured to transmit light to the optical sensor. The light source 206 may, for example, include a laser that emits a continuous wave or pulsed laser energy into the sensor via an optical waveguide such as an optical fiber. In variations in which the optical sensor is a WGM resonator, the light from the light source 206 may propagate a first set of WGMs around a surface (e.g., wall) of the circumference of the WGM resonator. Propagation of the first set of WGMs may result in generation of a first set of optical signals corresponding to a first set of resonant frequencies of the WGMs. As the temperature and/or pressure in a measurement region proximate to the WGM resonator changes, a set of changes to the radius and/or the refractive index of the WGM resonator may be induced, and/or the refractive index of an environment proximate to the WGM resonator. In some variations, these set of changes can propagate a second set of WGMs around the wall of the circumference of the WGM resonator. Propagation of the second set of WGMs may result in generation of a second set of optical signals corresponding to a second set of resonant frequencies of the WGMs. In some variations, the first set of optical signals and the second set of optical signals can be configured to propagate in the optical waveguide 204 to the photodetector 208, which then processes the optical signals into sensor responses (e.g., converts the first set of optical signals into a first set of electrical signals, and converts the second set of optical signals into a second set of electrical signals).

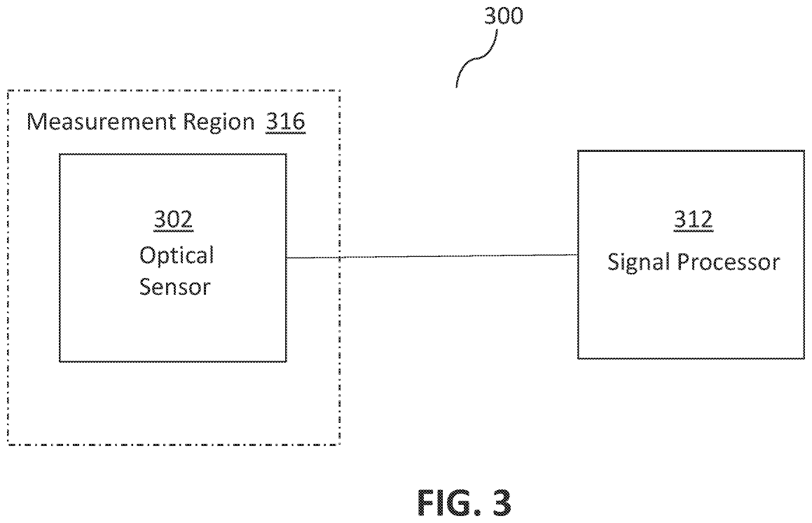

In some variations, the photodetector 208 may be coupled to one or more processors configured to generate measurement signals corresponding to a plurality of physical signals, based on a plurality of sensor responses determined from a sensor signal (e.g., electrical signals from the photodetector 208). In some variations, one or more of the processors may be part of a signal processor such as that described below with respect to FIG. 3.

For example, as shown in FIG. 2, the photodetector 208 may be coupled to a temperature processor 210 and a pressure processor 212. The temperature processor 210 may be configured to characterize one or more sensor responses determined from the first set of electrical signals and/or the second set of electrical signals (e.g., sensor signals), and generate measurement signal(s) corresponding to temperature. In some variations, the temperature processor 210 may generate measurement signal(s) indicating a change in temperature and/or an absolute value of the temperature based on a sensor response among the first and second sets of electrical signals. For example, a mode shift (e.g., change from the first set of resonant frequencies of WGMs in the first set of optical signals to the second set of resonant frequencies of WGMs in the second set of optical signals) may correspond to or be indicative of the temperature of the measurement region. Accordingly, the temperature processor 210 may be configured to generate temperature information of the measurement region by analyzing the mode shift. Similarly, baseline drift (e.g., a change in baseline signal value(s) of the first set of optical signals to the second set of optical signals) may correspond to or be indicative of the pressure of the measurement region. Accordingly, the pressure processor 212 may be configured to generate pressure information of the measurement region by analyzing the baseline drift. In some variations, mode shift may include a shift in resonant frequencies. However, it should be understood that mode shift may include one or more of a shift in frequencies, a shift in the wave height (e.g., depth change), and a shift in the shape of the wave (e.g., shape change).

In some variations, the plurality of measurement signals corresponding to a plurality of physical signals (e.g., temperature information and the pressure information) may be transmitted, for example, to the display 214 for real-time monitoring of the measurement region. In some variations, the display 214 may include an interactive user interface (e.g., a touch screen) and may be configured to transmit a set of commands (e.g., pause, resume, and/or the like) to the light source 206. In some variations, the system 200 may further include a set of ancillary devices (not shown) configured to input information to the system 200 or output information from the system 200. In some variations, the set of ancillary devices may include one or more of a keyboard, a mouse, a monitor, a webcam, a microphone, a touch screen, a printer, a scanner, a virtual reality (VR) head-mounted display, a joystick, a biometric reader, and the like. Additionally or alternatively, in some variations, the system 200 may include or be communicatively coupled to one or more storage devices (e.g., local or remote memory device(s)).

It should be readily understood that although FIG. 2 illustrates two separate processors (i.e., temperature processor 210 for measuring temperature and pressure processor 212 for measuring pressure), in some variations a single processor may be used to extract the temperature information and pressure information. Furthermore, in some variations multiple processors may be used to extract temperature information, and/or multiple processors may be used to extract pressure information (e.g., for purposes of averaging different measurement signals). In other words, there may be a n:m ratio of processors to measured physical signals where n is equal to m, or n is less than m, or n is greater than m. Furthermore, processor(s) may be configured to determine other kinds of measurement data corresponding to different physical signals. For example, in some variations a third processor (not shown) may be configured to determine measurement data from sensor responses corresponding to acoustic waves (e.g., ultrasound imaging).

FIG. 3 is a schematic of an exemplary variation of a system 300 for sensing a plurality of physical signals. In some variations, the system 300 may include an optical sensor 302 (e.g., WGM resonator) comprising a measurement region 316 proximate to the optical sensor 302 and a signal processor 312. The optical sensor 302 may be communicatively coupled to the signal processor 312 via a photodetector (not shown in FIG. 3), similar to that described with respect to FIG. 2. For example, similar to that described above, the optical sensor 302 may be optically coupled to the optical detector. The optical detector in turn may be communicatively coupled to the signal processor 312 via a network.

The optical sensor 302 may be any suitable optical sensor and may include any of the optical resonators (e.g., WGM resonator) described herein. The measurement region 316 may include a polymer structure packaging the WGM resonator.

In some variations, the signal processor 312 may include one or more processors (e.g., CPU) (e.g., similar to the processors described above with respect to FIG. 2). The processor(s) may be any suitable processing device configured to run and/or execute a set of instructions or code, and may include one or more data processors, image processors, graphics processing units, digital signal processors, and/or central processing units. The processor(s) may be, for example, a general purpose processor, a Field Programmable Gate Array (FPGA), an Application Specific Integrated Circuit (ASIC), and/or the like. The processor(s) may be configured to run and/or execute application processes and/or other modules, processes and/or functions associated with the system 300.

In some variations, the signal processor 312 may run and/or execute application processes and/or other modules. These processes and/or modules when executed by a processor may be configured to perform a specific task. These specific tasks may collectively enable the signal processor 312 to analyze sensor responses to generate multiple measurement signals, each of which may be indicative of a physical signal. For example, these specific tasks may enable the signal processor 312 to detect multiple physical signals accurately based on sensor responses of the optical sensor 302 caused by changes to temperature, pressure, acoustic wave(s), and the like in the measurement region 316.

In some variations, application processes and/or other modules may be software modules. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the signal processor 312 may comprise a memory configured to store data and/or information. In some variations, the memory may comprise one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

In response to changes in the measurement region 316, the optical sensor 302 may be configured to send one or more sensor signals (e.g., mode shift, baseline drift, mode split, mode broadening) as optical signals to an optical detector. The optical detector may be configured to convert the optical signals into a plurality of sensor responses (e.g., electrical signals). The electrical signals indicative of the sensor responses may be transmitted to the signal processor 312. In some variations, the optical detector (not shown in FIG. 3) may be communicatively coupled to the signal processor 312 via a computing device and/or a network (not shown in FIG. 3).

In some variations, the signal processor 312 may be configured to analyze the sensor responses using methods such as those described below (e.g., by decoupling or otherwise differentiating between individual physical signals, and/or collectively analyzing the multiple sensor responses to determine individual physical signals). The signal processor 312 may be further configured to generate measurement signals that may correspond to (e.g., be indicative of) the individual physical signals. In this manner, the signal processor 312 may be configured to detect a plurality of physical signals.

### Methods for Detecting Multiple Physical Signals

In some variations, a method for detecting multiple physical signals may include receiving a sensor signal from a single optical sensor proximate to a measurement region. Multiple sensor responses may be determined from the sensor signal. Multiple measurement signals may be generated from the multiple sensor responses, where each measurement signal may be indicative of a different physical signal of the measurement region. These multiple measurement signals may be generated by various techniques, such as decoupling individual physical signals and/or collectively analyzing the multiple sensor responses to determine individual physical signals, as further described below.

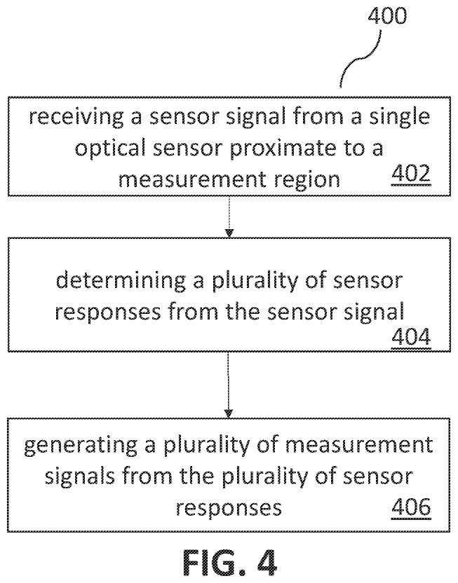

FIG. 4 is a flowchart of an exemplary variation of a method 400 for detecting multiple physical signals using a single optical sensor. Method 400 may include receiving a sensor signal from a single optical sensor proximate to a measurement region (402), determining a plurality of sensor responses from the sensor signal (404), and generating a plurality of measurement signals from the plurality of sensor responses (406). Each measurement signal may be indicative of a different respective physical signal of the measurement region. The method may be performed, for example, by a signal processor (e.g., structurally and/or functionally similar to signal processor 312 in FIG. 3) that may receive a sensor signal from a single optical sensor (e.g., structurally and/or functionally similar to optical sensor 102 in FIG. 1, 202 in FIG. 2, or 302 in FIG. 3) proximate to a measurement region.

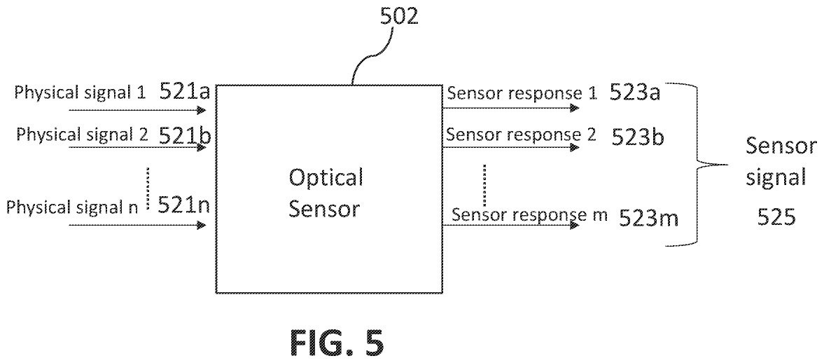

FIG. 5 illustrates a schematic of an exemplary variation of an optical sensor 502 for detecting multiple physical signals. Physical features of a measurement region proximate to the optical sensor 502 may be reflected in the sensor responses of the optical sensor 502. For example, the optical sensor 502 may be configured to detect physical features (e.g., physical signals) of the measurement region such as acoustic waves (e.g., reflected acoustic waves), temperature, and pressure. These features are depicted as physical signals 521a, 521b, 521n, etc. (generally referred to as physical signal 521). In response to the physical signals, the optical sensor 502 may generate a sensor signal 525 comprising one or more sensor responses such as a mode shift, a baseline drift, a mode split, a mode broadening, and the like. These sensor responses are depicted as sensor response 523a, 523b, 523m, etc. (generally referred to as sensor response 523). The sensor signal 525 may comprise all the sensor responses 523a, 523b, 523m, etc., outputted by the optical sensor 502.

The detection of multiple physical signals by the optical sensor 502 may be described as:

$${y}_{i} = {T}_{i} ( {x}_{1} , {x}_{2} , … , {x}_{n} ) , i = 1 , 2 , … m \qquad ( 1 )$$

where {xᵢ} represents physical signals, {yᵢ} represents sensor responses, and {Tᵢ} represents system transformations. In general, either xᵢ or yᵢ may be a function of time, but may also be a simple variable. Tᵢ may be either linear or nonlinear.

In some variations, each sensor response 523 may be associated with (e.g., more sensitive to or responsive to) a predetermined physical signal 521. For example, sensor response 523a may be more sensitive to physical signal 521a while sensor response 523b may be more sensitive to (e.g., more closely associated with) physical signal 521b. In some variations, a plurality of sensor responses may be associated with a predetermined physical signal.

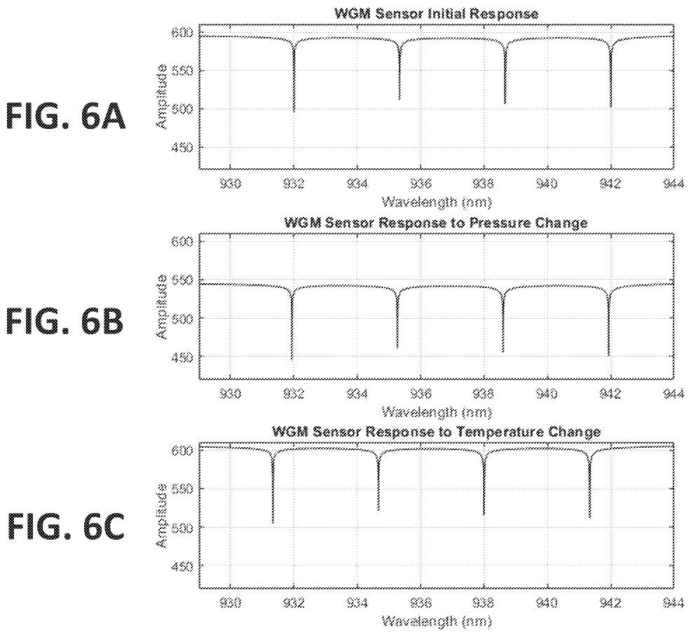

As an illustrative example of how different sensor responses may be associated with different physical signals, FIGS. 6A-6C depict plots illustrating three example sensor signals generated by an exemplary WGM resonator in response to different physical signals (temperature and pressure) for a measurement region. The horizontal axis of each plot represents wavelength in nm and the vertical axis represents the amplitude of the WGM resonator optical output.

FIG. 6A depicts a sensor signal under conditions of the measurement region where temperature T=t₀ and pressure P=p₀. As seen in this plot, the sensor signal may have two sensor responses, one relating to resonant frequencies and one relating to a baseline value. For example, the sensor signal may have a first sensor response representing four resonant frequencies at about 932 nm, about 935.3 nm, about 938.7 nm, and about 942 nm locations, with a second sensor response representing a baseline around 590 units amplitude.

FIG. 6B depicts the sensor signal in response to a pressure change in the measurement region: P=p₀+Δp. As seen in this plot, the baseline (e.g., sensor response) is shifted significantly to around 540 units of amplitude relative to the baseline amplitude shown in FIG. 6A. However, the resonant frequencies have barely decreased relative to that shown in FIG. 6A. Accordingly, FIG. 6B indicates that baseline shift is a sensor response that is more sensitive to pressure than mode shift. In other words, a baseline of a WGM resonator response may be more suitable for detecting pressure variation. In some variations, the sensor response (e.g., baseline) may be represented as a percentage of change in the sensor response as the pressure changes. For example, the baseline may be represented as a percentage of change in the baseline from P=p₀ to P=p₀+Δp.

FIG. 6C represents the sensor signal in response to a temperature change in the measurement region: T=t₀+Δt. In FIG. 6C, the resonant frequencies are each significantly decreased by more than 0.7 nm (e.g., at least about 0.007%) relative to that shown in FIG. 6A, while the baseline has only very slightly increased to around 600 units of amplitude relative to that shown in FIG. 6A. Accordingly, FIG. 6C indicates that mode shift is a sensor response that is more sensitive to temperature than baseline shift. In other words, resonant frequency shift may be more suitable for detecting temperature fluctuation. In some variations, the sensor response (e.g., frequency shift) may be represented as a percentage of change in the sensor response as the pressure changes. For example, the mode shift may be represented as a percentage of change in mode shift from T=t₀ to T=t₀+Δt.

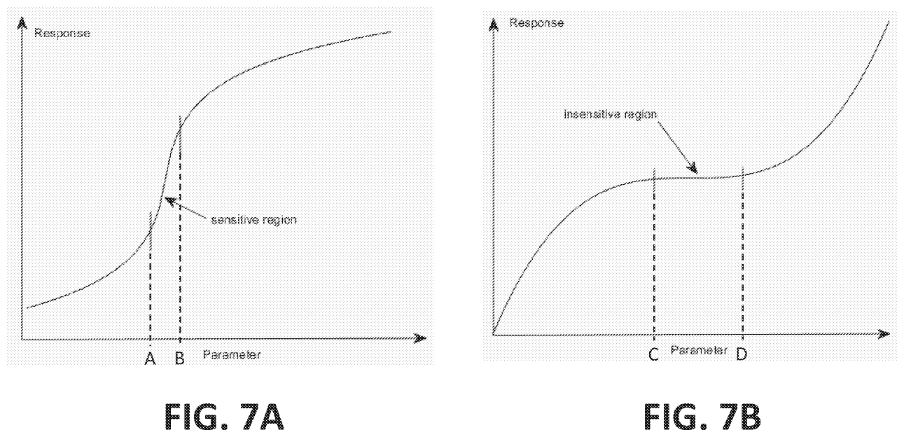

In addition to being more sensitive to a particular physical signal, a sensor response may have at least one sensitive region for that physical signal, in that the sensor response may have a region in which the sensor response varies particularly rapidly (relative to a predetermined slope threshold) in response to change in the physical signal. FIG. 7A is an illustrative plot showing an exemplary sensor response curve with a sensitive region. As seen in FIG. 7A, at lower values of a physical signal below value A, the sensor response increases slowly with increase in the physical signal. However, in the sensitive region of the optical sensor (the segment of the sensor response curve indicated by the two vertical bars corresponding to parameter values A and B of the physical signal), the slope of the sensor response curve becomes steeper, indicating that the sensor response increases more rapidly per unit increase in the physical signal between values A and B of the physical signal. At higher values of the physical signal above value B, the sensor response again increases slowly with an increase in the physical signal.

Additionally or alternatively, a sensor response may have at least one insensitive region for a physical signal, in that the sensor response may have a region in which the sensor response varies relatively slowly in response to change in the physical signal. FIG. 7B is an illustrative plot showing an exemplary sensor response curve having an insensitive region. As seen in FIG. 7B, at lower values of a physical signal below value C, the sensor response increases rapidly with increase in the physical signal. However, in the insensitive region of the optical sensor (the segment of the sensor response curve indicated by the two vertical bars corresponding to parameter values C and D of the physical signal), the slope of the sensor response curve is about zero, indicating that the sensor response has little to no change per unit increase in the physical signal. At higher values of the physical signal above value D, the sensor response again more rapidly increases with the increase in physical signal.

Accordingly, as described above, the method for multi-dimensional sensing may include generating measurement signals from the sensor responses, where each of these measurement signals may be indicative of a respective physical signal. For example, a signal processor may generate a temperature measurement signal based at least in part on the resonant frequency shift (e.g., mode shift) and a pressure measurement signal based at least in part on baseline drift. Described in further detail below are example variations of generating measurement signals from sensor responses, including decoupling individual physical signals and/or collectively analyzing the multiple sensor responses to determine individual physical signals.

### Decoupling Individual Physical Signals

As discussed above, each sensor response may be relatively more sensitive to a predetermined physical signal. Accordingly, in some variations, measurement signals for different physical signals may be generated by decoupling individual physical signals, where decoupling may include analyzing each sensor response for a predetermined time period and generating a measurement signal for the physical signal that correlates the most to that sensor response. As such, the decoupled physical signals may be distinguished from one another, and analyzed separately by analyzing the decoupled (e.g., separated) sensor responses.

Furthermore, as described above, a sensor response may be relatively more sensitive to a particular physical signal (e.g., mode shift may be sensitive to temperature, baseline drift may be more sensitive to pressure). Accordingly, a method for sensing multiple physical signals may include associating one sensor response to one physical signal. For example, the method may include correlating mode shift to temperature measurements and correlating baseline drift to pressure measurements. Following this association, the sensor response (output of the optical sensor) may be selected for processing such that a sensor response being analyzed correlates to a target physical signal (e.g., input to the photo detector).

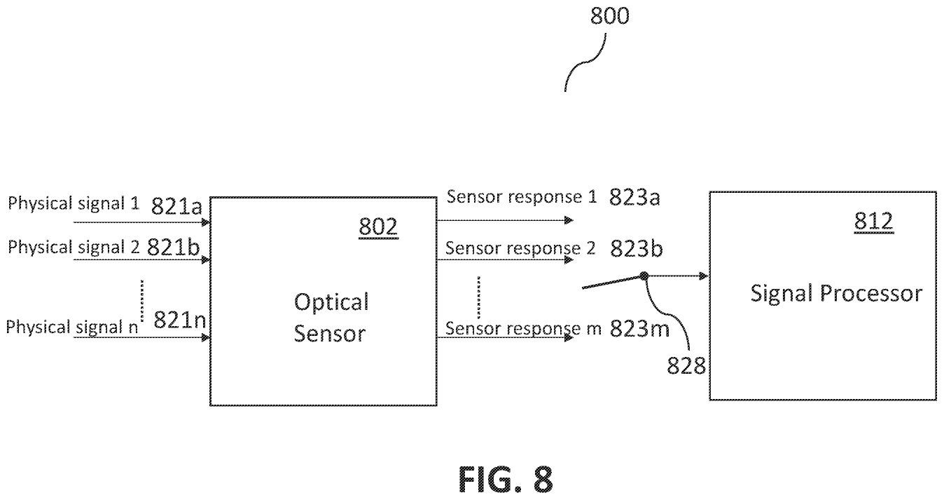

For example, FIG. 8 is an illustrative schematic of a system 800 for decoupling individual physical signals. The system 800 may comprise an optical sensor 802 (e.g., structurally and/or functionally similar to optical sensor 102 in FIG. 1, 202 in FIG. 2, or optical sensor 302 in FIG. 3) communicatively coupled to a signal processor 812 (e.g., structurally and/or functionally similar to signal processor 312 in FIG. 3).

The system may also include a multiplexer 828 configured to control selection and output of one or more sensor responses from the set of sensor responses (e.g., collectively referred to as a sensor response) to the signal processor 812. In some variations, the multiplexer 828 may comprise a time division multiplexer. Accordingly, the multiplexer 828 may be configured to selectively connect an individual sensor response channel to the signal processor 812 for each time period. For example, the multiplexer 828 and the signal processor 812 may be configured to output a first sensor response during a first time period and a second sensor response during a second time period. The signal processor 812 may additionally be configured to output a third sensor response during a third time period, and so on.

Therefore, although the optical sensor 802 may be configured to generate more than one sensor response, owing to the multiplexer 828, the signal processor 812 may be configured to process only one sensor response at any given time. For instance, in FIG. 8, the signal processor 812 may be configured to process sensor response 823a between time t₀ and t₁, sensor response 823b between time t₁ and t₂, and sensor response 823m between time tₘ₋₁ and tₘ. Consider that the sensor response 823a may be the most sensitive to physical signal 821a, sensor response 823b may be the most sensitive to physical signal 821b, and sensor response 823m may be the most sensitive to physical signal 821n. Accordingly, the signal processor 812 may be configured to generate measurement signals for physical signal 821a throughout time t₀ and t₁, measurement signals for physical signal 821b throughout time t₁ and t₂, and measurement signals for 823m throughout time Iₘ₋₁ and Iₘ. In some variations, after each of the sensor responses are processed at least once for a predetermined duration, the signal processor 812 may be configured to process the first sensor response 823a again and continue to analyze the responses until the decoupling process ends. In some variations, the signal processor 812 may be configured to process the sensor responses in one of at least two modes-continuous mode/endless mode or finite duration mode. In the continuous mode, each of the sensor responses may be processed at least once and repeatedly (e.g., a repeated series of sensor responses 823a-823m consecutively, or in any suitable series) until the signal processor 812, the optical sensor 802, and/or the system 800 is shut down (e.g., by a user). In the finite duration mode, the signal processor 812 may be configured to analyze the sensor responses for a predetermined amount of time. For example, all of the sensor responses may be processed at least once and the signal processor 812 may be configured to process the first response 823a again and continue to analyze the responses until the predetermined amount of time (e.g., 2 hours or other suitable period of time, which may additionally or alternatively be expressed as a predetermined number of times that each sensor response is processed) has elapsed. In this manner, the signal processor 812 may be configured to decouple the individual physical signals using time division multiplexing. Given a sufficiently high switching speed of the multiplexer, it may be possible to detect multiple physical signals in real-time or near real-time without losing information.

In some variations, the sequence of selecting the individual sensor response for handling by the signal processor may be predetermined. For example, the signal processor may alternate between two sensor responses, or may repeatedly handle three or more sensor responses in the same sequential order. As another example, certain physical signals that are known to inherently have a slower rate of change may be sampled at a low rate for handling (e.g., in some applications for certain kinds of measurement regions, it may be known that temperature of the measurement region change cannot fluctuate as quickly as pressure). As another example, in some applications, certain physical signals whose values are more critical to measure in real-time may be sampled at a higher rate for handling, compared to less critical physical signals, in order to prioritize the more critical physical signals for measurement.

Additionally or alternatively, the time period (e.g., duration of analysis) over which each sensor response is processed by the signal processor may be predetermined. For example, it may be predetermined that all sensor responses may be processed for equal periods of time. As another example, certain physical signals that are known to inherently have a slower rate of change or that (in at least some application) are less critical may be processed for shorter periods of time.

Additionally or alternatively, the sequence of selecting the individual sensor response for processing by the signal processor and/or the amount of time over which each sensor response may be processed may be at least sometimes determined by the signal processor 812 in real-time. In some variations, the sequence in which sensor responses are processed and/or the amount of time that each sensor response is processed may be based at least in part on a closed-loop algorithm. For example, rate of change of a physical signal over a particular period of time may be fed into a closed-loop algorithm to dynamically adjust the frequency at which the signal processor handles the sensor response corresponding to that physical signal, and/or the overall amount of time during which the signal processor handles the sensor response corresponding to that physical sensor. For example, in response to a physical signal that has recently started to change rapidly, the signal processor may handle the sensor response corresponding to that physical signal more frequently and/or for longer periods of time in order to monitor that physical signal more closely to real-time.

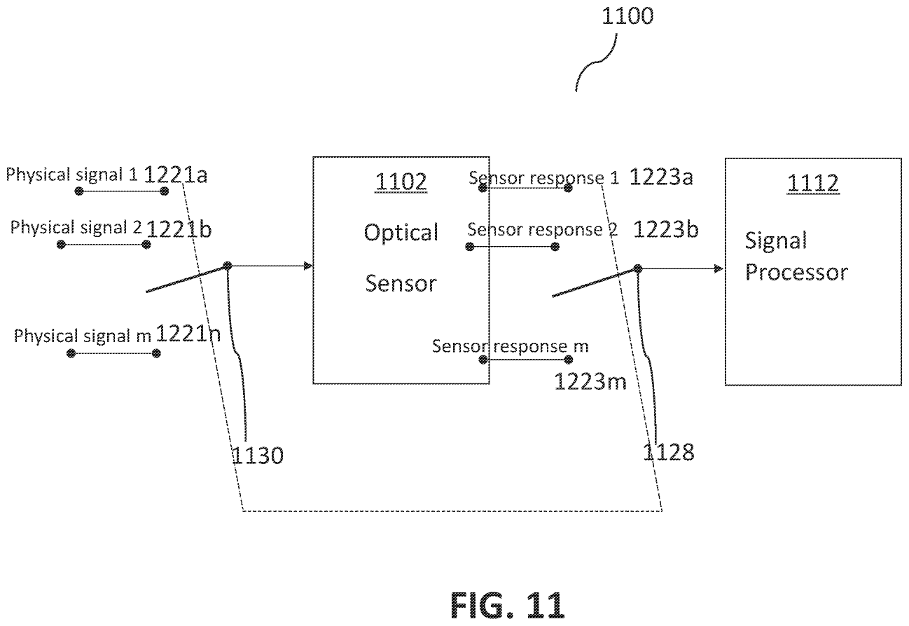

FIG. 11 is another exemplary variation of a system 1100 for distinguishing between physical signals. The system 1100 may comprise an optical sensor 1102 (e.g., structurally and/or functionally similar to optical sensor 102 in FIG. 1, optical sensor 202 in FIG. 2, or optical sensor 302 in FIG. 3) communicatively coupled to a signal processor 1112 (e.g., structurally and/or functionally similar to signal processor 312 in FIG. 3).

The system may also include a plurality of multiplexers, for example, a first multiplexer 1128 (e.g., structurally and/or functionally similar to multiplexer 828) and a second multiplexer 1130. In some variations, the multiplexers may be synchronized. For instance, if the target physical signal to be measured is temperature, then the second multiplexer 1130 may be switched to detect temperature and the first multiplexer 1128 may be synchronized with the second multiplexer 1130 to select the sensor response mode shift for processing/analysis. However, if the target physical signal to be measured in pressure, then second multiplexer 1130 may be switched to detect pressure and the first multiplexer 1128 may be synchronized with the second multiplexer 1130 to select the sensor response baseline drift for processing/analysis. In this manner, correlating the sensor response to the physical signal and using a multiplexer to select the target physical signal and the target sensor response may reduce and/or minimize interference from other physical signals and enable the system 1100 to accurately measure the target physical signal.

In some variations, a sensor response may be correlated to a physical signal based at least in part on other known information. For example, in some variations multiple possible physical signals may contribute to a particular sensor response, but relative sensitivities of an optical sensor to those physical signals may be known, and may be used to help distinguish or differentiate between different physical signals. For example, an optical sensor may inherently have a sensor response (e.g., mode shift) to both temperature and acoustic waves, but differing known sensitivities to each of these physical signals due to the nature of the environment around the sensor. The temperature signal may be differentiated from the acoustic wave signal based on, for example, a rate of change in sensor response and the sensor's relative sensitivities to temperature and pressure. For instance, if the optical sensor is placed in a thermally insulated environment and is thereby somewhat insensitive to temperature change, then rapid changes to mode shift may be more indicative of an acoustic wave signal than a temperature signal. In contrast, if the optical sensor is adjacent to or embedded in a damping material, then rapid changes to mode shift may be more indicative of a temperature signal than an acoustic wave signal.

Additionally or alternatively, in some variations, other known information that is used to help distinguish between different physical signals may include typical frequency ranges of sensor response. For example, one or more predetermined thresholds for frequency of sensor response may be used to help distinguish between different physical signals. As an illustrative example, as described above, an optical sensor may inherently have a sensor response (e.g., mode shift) to both temperature and acoustic wave. Accordingly, in some variations, a mode shift (e.g., frequency shift, change in depth, change in shape) that involves frequencies above a predetermined threshold (e.g., above about 500 kHz) may be more indicative of an acoustic wave signal than a temperature signal. In contrast, a mode shift that involves frequencies below a predetermined threshold (e.g., below about 500 kHz) may be more indicative of a temperature signal than an acoustic wave signal. In some variations, a single predetermined threshold may be used to distinguish between physical signals (e.g., sensor response having a characteristic above the threshold corresponds to or is indicative of a first physical signal, sensor response having a characteristic below the same threshold corresponds to or is indicative of a second physical signal). Alternatively, in some variations multiple predetermined thresholds may be used to distinguish between physical signals. For example, a sensor response having a characteristic above a first threshold may correspond to (e.g., be indicative of) a first physical signal, and a sensor response having a characteristic below a second threshold that is lower than the first threshold may indicate a second physical signal. However, a sensor response having a characteristic between the first and second thresholds may be treated as indicative of either the first or second physical signal, and further analysis (e.g., one of the other methods described herein) may be used to further distinguish between the first and second physical signals.

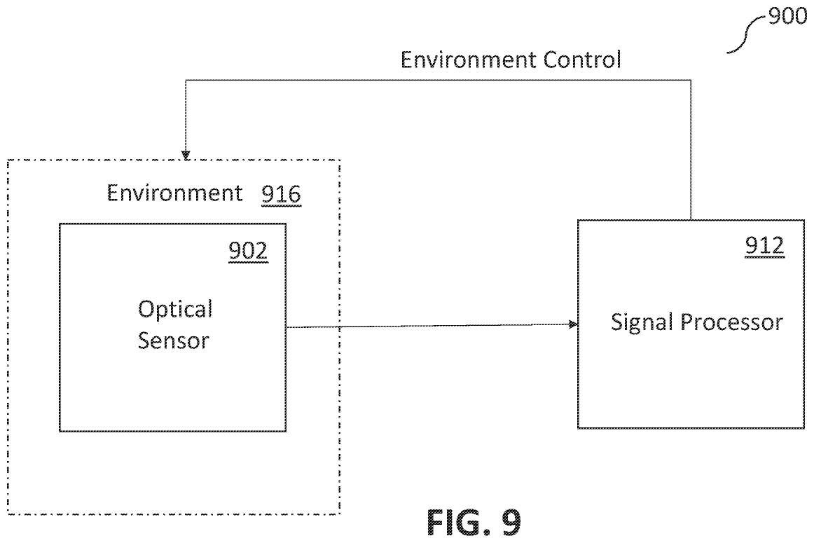

In some variations, decoupling the individual physical signal may additionally or alternatively include altering the environment proximate to the optical sensor so as to selectively enhance the prominence of a sensor response associated with a target physical signal, either by making the environment more sensitive to the target physical signal, or less sensitive to all physical signals other than the target physical signal. FIG. 9 is an exemplary variation of a system 900 for decoupling individual physical signals by altering an environment of an optical sensor. The system 900 may comprise an optical sensor 902 (e.g., structurally and/or functionally similar to optical sensor 102 in FIG. 1, optical sensor 202 in FIG. 2, or optical sensor 302 in FIG. 3) that is in an environment 916 (e.g., polymer packaging, other surrounding packaging) and is communicatively coupled to a signal processor 912 (e.g., including a signal processor structurally and/or functionally similar to signal processor 312 in FIG. 3). The optical sensor 902 may be proximate to an environment 916 (e.g., structurally and/or functionally similar to measurement region 316 in FIG. 3).

In FIG. 9, the signal processor in the signal processor 912 may first process all sensor responses corresponding to physical signals, analyze these sensor responses, and generate an environmental control signal for altering one or more physical properties of the environment based on the identity of at least one target physical signal. For example, the signal processor 912 may provide instructions to the system to alter the environment 916 such that one or more physical signals that are different from the target physical signal are suppressed. For example, suppression of a physical signal may comprise adjusting the environment such that the physical signal (that is not being targeted for measurement) is within a relatively low sensitivity signal region of the optical sensor. The signal processor may then process the sensor responses from the altered environment to generate measurement signals for the targeted physical signal(s), which are more prominent in view of the suppression of untargeted physical signal(s).

For example, in order to make the physical signal for temperature more prominent to enable more accurate temperature measurements, it may be advantageous to suppress the physical signal for pressure by reducing or eliminating undesired pressure fluctuations in the sensor environment 916. Reduction or elimination of environment pressure change may be performed, for example, by detecting current pressure and then adjusting the environment pressure accordingly in a feedback manner (e.g., moving the environment pressure level to be within the insensitive region of the baseline sensor response for the optical sensor) using the signal processor 912. When the environment pressure is in the insensitive region, the signal processor may generate a measurement signal based on the sensor response associated with temperature (e.g., mode shift). The manner of modulation of environment pressure may depend on the construction of the sensor package. For example, in some variations an optical sensor may be disposed within a cavity (or between two plates), and fluid (gas, liquid) may be selectively introduced and/or withdrawn from the cavity using one or more controllable fluidic valves and/or one or more pressurized sources (e.g., positive pump, vacuum pump). In this example, environment pressure may be increased by the introduction of additional fluid into the cavity, and/or reduced by the withdrawal of fluid from the cavity. As another example, in some variations an optical sensor may be disposed within a cavity (or between two plates) and surrounded by a fluid (gas, liquid), where the cavity volume is adjustable by one or more actuators. In this example, the environment pressure may be increased by reduction of cavity volume (e.g., moving a cavity wall inwards, compressing a deformable cavity) and/or decreased by increasing cavity volume (e.g., moving a cavity wall outwards).

As another example, in order to make the physical signal for pressure more prominent to enable more accurate pressure measurements, it may be advantageous to suppress the physical signal for temperature by reducing or eliminating undesired temperature fluctuations in the sensor environment. Undesired temperature fluctuations may be eliminated and/or reduced, for example, by adjusting the environment 916 to move its temperature levels to the insensitive region of the mode shift sensor response for the optical sensor with the signal processor 912. For example, the signal processor 912 may provide feedback/instructions to alter the environment such that the temperature levels may be moved to the insensitive region. When the temperature level reaches the insensitive region, the signal processor may measure the pressure and/or extract information relating to pressure from the sensor response correlated to pressure (e.g., baseline drift). The manner of modulation of environment temperature may depend on the construction of the sensor package. For example, in some variations the temperature of the surrounding environment of the sensor may be reduced by thermally insulating the surrounding environment (e.g., by using thermal insulation materials such as glass fiber, mineral wool, polyurethane, or by passing a fluid with thermally insulating properties in contact with or near the sensor). As another example, in some variations, the temperature of the surrounding environment may be increased by using thermally conductive material (e.g., copper, graphite, or by passing a fluid with thermally conductive properties in contact with or near the sensor) for the surrounding environment.

As another example, in some variations it may be advantageous to alter an acoustic property of the environment of the sensor, such that a physical signal related to acoustic waves (e.g., acoustic waves reflected from the measurement region) is suppressed, which may allow another targeted physical signal (e.g., temperature, pressure) to be more measured more accurately. For example, a suitable actuator may move a damping material toward or near an optical sensor such that incoming acoustic waves that would otherwise be detected are instead attenuated (e.g., suppressed).

Additionally or alternatively, it may be possible to increase sensitivity of the optical sensor to certain input physical signals (e.g., “tuning” the optical sensor to a targeted physical signal). For example, the sensitivity of the optical sensor to a target physical signal may be increased at least in part by adjusting an environment of the sensor such that the target physical signal is in a relatively higher sensitivity signal region of the optical sensor.

For example, the sensitivity of pressure detection may be increased by adjusting the environment 916 such that the pressure levels in the environment 916 fall within the sensitive region of the baseline drift curve. As discussed above, in some variations baseline drift is the sensor response that is most sensitive to changes in pressure. Accordingly, the signal processor 912 may provide feedback/instructions so that the pressure levels in the environment are altered to fall within the sensitive region of the sensor's response curve. The manner of modulation of environment pressure may depend on the construction of the sensor package. For example, any of the above-described techniques (e.g., control of fluid in and/or out of a cavity including the sensor, controlling volume of a fluid-filled cavity including the sensor) may be implemented to modulate environment pressure such that it lies in the sensitive region of the optical sensor and the optical sensor is tuned to measure pressure of a measurement region with greater sensitivity. Thus, once the pressure level is in the sensitive region, the signal processor may measure the pressure and/or extract information (e.g., relating to pressure from the baseline drift).

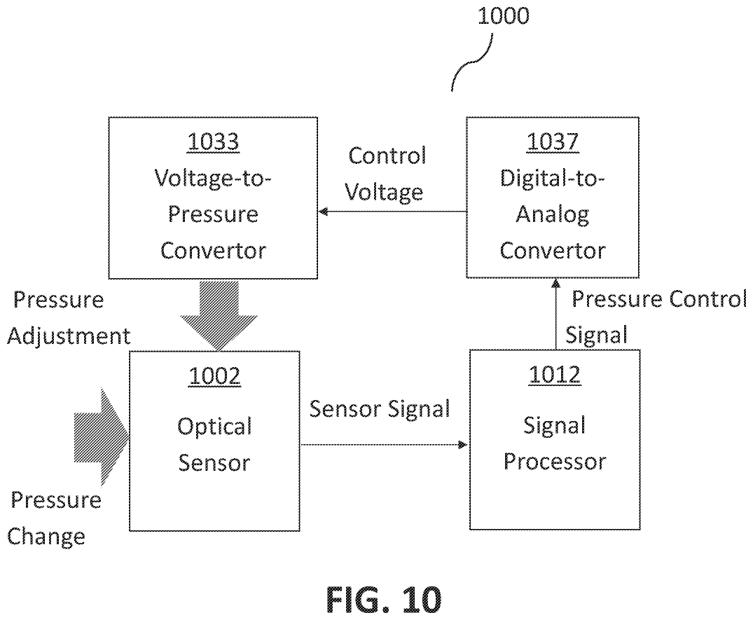

FIG. 10 illustrates an exemplary variation of a system 1000 for altering an environment by adjusting a pressure of the environment. In some variations, the system 1000 may comprise an optical sensor 1002, a signal processor 1012, a digital-to-analog convertor 1037, and a voltage-to-pressure convertor 1033. The optical sensor 1002 (e.g., structurally and/or functionally similar to the optical sensor 102 in FIG. 1, the optical sensor 202 in FIG. 2, or the optical sensor 302 in FIG. 3) may be communicatively coupled to a signal processor 1012 (e.g., structurally and/or functionally similar to signal processor 312 in FIG. 3). The optical sensor 1002 may be configured to generate a sensor signal after detecting a pressure change in the environment and may transmit the sensor signal to the signal processor 1012. In some variations, the signal processor 1012 may be configured to process the sensor signal to generate a digital control instruction. In some variations, the digital control instruction may include instructions to alter the environment by adjusting a pressure level so as to increase or decrease the sensitivity of the optical sensor 1002 for accurate pressure or temperature measurement. The digital-to-analog converter 1037 may be configured to convert the digital control instruction to an analog voltage signal. The analog voltage signal may be transmitted to the voltage-to-pressure converter 1033. In some variations, the voltage to pressure converter 1033 may be configured to generate the desired pressure (e.g., the target pressure included in the digital control instruction) so as to increase or decrease the sensitivity of the optical sensor 1002 for accurate pressure or temperature measurement.

As discussed above, if the system 1000 is incorporated in an application that requires accurate sensing of temperature, then it may be advantageous to adjust the pressure within the insensitive region. However, if the system 1000 is incorporated in an application that requires accurate sensing of pressure, then it may be advantageous to adjust the pressure within the sensitive region. For example, in order to enhance sensitivity to adjust the pressure within the sensitive region, pressure may be increased in the same direction as the pressure change. For instance, if the pressure in the environment is increased from 100 to 101 KPa, an additional 2 KPa pressure may be added to the pressure in the environment to generate a 103 KPa pressure so that the pressure falls within the sensitive region. If, however, the sensitivity changes such that the pressure is within the insensitive region, pressure may be reduced to follow the direction of the pressure change. For instance, if the pressure in the environment is decreased from 100 to 98 KPa, an additional 1 KPa of pressure may be added to the pressure in the environment to generate a 99 KPa pressure. In some variations, the pressure in the environment may be kept at a constant 100 KPa.

Similarly, as another example, referring back to FIG. 9, the sensitivity of temperature detection may be increased by adjusting the environment 916 such that the temperature of the environment is within the sensitive region of a mode shift curve. As discussed above, in some variations, mode shift is the sensor response that is most sensitive to changes in temperature. Accordingly, the signal processor 912 may be configured to provide feedback/instructions so that the temperature of the environment is altered to be in the sensitive region of the sensor's response curve. The manner of modulation of temperature may depend on the construction of the sensor package. For example, any of the above-described systems or techniques may be used to heat or cool the sensor environment toward a sensitive region of the sensor (e.g., using thermally insulating materials or thermally conductive materials).

Additionally or alternatively, in some variations, detection of acoustic waves may be selectively enhanced by adjusting the environment such that one or more acoustic properties of the environment is improved or optimized. For example, in some variations a suitable actuator may be configured to move an acoustic matching material toward or near an optical sensor to improve transmission and/or reduce attenuation of incoming acoustic waves that otherwise might be difficult to detect.

Accordingly, decoupling may include selectively altering the environment based on the targeted physical signal to be measured. As discussed above, altering the environment may include suppressing a physical signal that is not the targeted physical signal by adjusting the physical signal to be suppressed within the insensitive region. Alternatively, altering the environment may include increasing the sensitivity of the targeted physical signal. Put differently, the environment may be adjusted such that the targeted physical signal may be within the sensitive region.

### Collectively Analyzing Multiple Sensor Responses

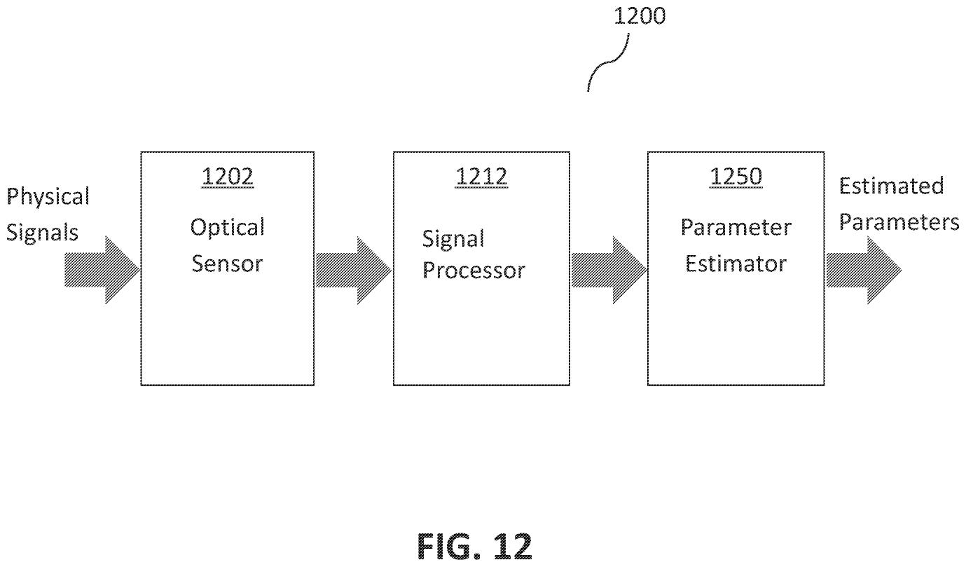

In some variations, multiple sensor responses may be collectively analyzed to determine measurement signals for individual physical signals. FIG. 12 is a schematic block diagram of an exemplary variation of a system 1200 for collectively analyzing multiple sensor responses to determine individual physical signals. The system may comprise an optical sensor 1202 (e.g., structurally and/or functionally similar to optical sensor 102 in FIG. 1, optical sensor 202 in FIG. 2, or optical sensor 302 in FIG. 3) communicatively coupled to a signal processor 1212 (e.g., structurally and/or functionally similar to signal processor 312 in FIG. 3). The signal processor 1212 may be coupled to a parameter estimator 1250 or may be included in the signal processor 1212.

In some variations, the optical sensor 1202 may convert n input physical signals into m different output response signals as described above. The signal processor 1212 may be configured to perform a series of signal processing steps including amplification, analog-to-digital conversion, and filtering of the sensor signal. The parameter estimator 1250 may be configured to estimate a set of target physical signal parameters using either theoretical or empirical models.

For example, solving for physical signals xᵢ using sensor responses yᵢ, where xᵢ and yᵢ are related by Equation 1 may represent the process of combining multiple responses. Alternatively, the physical signals xᵢ may be solved for as:

$${x}_{i} = {T}_{i}^{- 1} ( {y}_{1} , {y}_{2} , … {y}_{m} ) , i = 1 , 2 , … n \qquad ( 2 )$$

where Tᵢ⁻¹ represents reverse system transformations.

In some variations, it may be assumed that both xᵢ and yᵢ are simple variables, but can be real or complex. Additionally, it may be assumed that the system described by Eq. 1 in FIG. 5 is linear.

The first assumption is based on all the signals being either constants or slow-varying functions. Even for fast-varying functions, however, the assumption is still valid if the time interval is short enough. The second assumption is based on the system 1200 being stable and the ranges of all the signals being small enough to ensure linearity. For wide range signals, a wide non-linear range may be divided into several small linear regions.

With the above two assumptions, Eq. 1 can be rewritten as:

$${y}_{i} = {\sum }_{{j = 1}^{}}^{n} {a}_{ij} {x}_{j} , i = 1 , 2 , … m \qquad ( 3 )$$

where aᵢⱼ are coefficients determined by the multiple dimension sensor. Eq. 3 can be further simplified by using the following elegant matrix equation:

$$y = Ax \qquad ( 4 )$$

where x is an n dimensional vector, y is an m dimensional vector, and A is an m×n matrix.

There are three distinct scenarios in solving Eq. 4 depending upon the values of m and n. Each scenario requires a different approach and leads to different results.

Scenario 1: Underdetermined Systems. In this case, n>m, or there are more unknowns than equations. Underdetermined system has either no solution or infinitely many solutions. Therefore, this scenario may not be considered further because no useful result can be obtained for practical applications.

Scenario 2: Critical Systems. In this case, n=m, or there are the same number of equations as the number of unknowns. It can be mathematically proven that there is always a unique solution to a critical system if all the equations are completely independent of each other. The solution to a critical system can be obtained by

$$x = {A}^{- 1} y \qquad ( 5 )$$

where A⁻¹ is the inverse of A. If there exist at least two dependent equations, however, a critical system is reduced to an underdetermined system.

Scenario 3: Overdetermined Systems. In this case, n<m, or there are more equations than unknowns. According to the linear system theory, overdetermined systems have no solution in general, but may have solutions in some cases, for example some of the equations are not completely independent. An overdetermined system may become either a critical system or an underdetermined system depending up on the number of dependent equations it contains.

Although there are no exact solutions for most real overdetermined systems, approximate solutions can be obtained in several different ways. For example, using the least square method, an approximate solution can be obtained by

$$x = {( {A}^{T} A )}^{- 1} {A}^{T} y \qquad ( 6 )$$

where A^{T} is the transpose of A.

Based on the above discussion, detection of the physical signals or parameters may essentially be to determine matrices A, A⁻¹, A^{T} and (A^{T}A)⁻¹ A^{T}. The matrices may be computed either theoretically or empirically.

The theoretical approach may include three steps. First, a physical or theoretical model may be developed. Second, a set of equations may be derived using the model. Third, these equations may be linearized.

The empirical approach may also include three steps. First, a generic linear system model may be built. Second, experiments may be conducted for data generation. Third, coefficients of the system equations may be estimated using experimental data.

### Array of Optical Sensors

As described above, in some variations, a system for multi-dimensional sensing may include an array of two or more multi-dimensional optical sensors, each of which can perform multi-dimensional sensing as described herein. The methods disclosed herein can be extended from a single optical sensor to the array of optical sensors.

In some variations, each sensor in the array of optical sensors may be of a same type of optical sensor. For example, each sensor in the array may be an optical sensor that may be more sensitive to one type of physical signal (e.g., temperature, pressure, acoustic waves) over other types of physical signals. In such a scenario, the entire array of sensors may be configured to measure one physical signal with greater accuracy or precision. For example, the entire array of sensors may be configured to measure temperature with greater accuracy, or the entire array of sensors may be configured to measure pressure with greater accuracy. In some variations, having two or more optical sensors that are configured to target measurement of the same physical signal may be advantageous, such as for combining similar sensor signals together to “boost” sensor signal and/or system redundancy (e.g., fault tolerance, in case of sensor failure).

Alternatively, some sensors in the array of sensors may be configured to target measurement of a different physical signal than the rest of the sensors in the array of sensors (e.g., with different sensitivities to various physical signals such as by configuring the environment surrounding such sensors, as described above). For example, a first portion of the optical sensors may be configured to be more sensitive to a first physical signal (e.g., temperature), while a second portion of optical sensors may be configured to be more sensitive to a second physical signal (e.g., pressure). Furthermore, any suitable number of portions of the array may be configured to be more sensitive to any suitable physical signal (e.g., additionally a third portion of the optical array may be more sensitive to a third physical signal).

At least some optical sensors that are sensitive to the same physical signal may be grouped together (e.g., one line or cluster of sensors in the array may be sensitive to a first physical signal, a second line or cluster of sensors in the array may be sensitive to a second physical signal). Additionally or alternatively, at least some of the optical sensors that are sensitive to the same physical signal may be distributed equally or unequally (e.g., interspersed or alternating arrangement, or randomly distributed) that may, for example, allow greater area of sensing coverage for each physical signal.

Sensitivity of the optical sensors may be modulated in any suitable manner including those described herein, such as configuration of an environment surrounding each optical sensor to tune sensitivity of that optical sensor to one or various physical signals (e.g., to be more sensitive or less sensitive to a physical signal). Such configuration of the environment may be dynamic (e.g., dynamically adjusted in real-time in accordance with a desired measurement or sensing functionality), or may be built permanently into the design of the array (e.g., a first portion of the optical sensors always configured to be more sensitive to a first physical signal, a second portion of the optical sensors always configured to be more sensitive to a second physical signal).

In some variations, at least some of the optical sensors may be adjacent to (e.g., embedded in or otherwise proximate to) a surrounding environment that enhances a physical signal to be measured by those optical sensors. For example, the surrounding environment of at least some sensors may be thermally insulated and/or made thermally conductive so that the temperature of the surrounding environment falls within the sensitive region, such that these sensors are more sensitive to temperature.

In some variations, some sensors in the array (e.g., array of same type of sensors or different types of sensors) may have a surrounding environment that suppresses one or more physical signals other than a physical signal to be measured by those optical sensors. For example, some sensors may be shielded with a rigid barrier so that they may be shielded from pressure changes, and such sensors may, for example, be more sensitive to temperature signals. As another example, some sensors may be shielded with a damping barrier to shield the sensors from acoustic waves, and such sensors may, for example, be more sensitive to pressure signals.

In some variations, the system may include one or more reference sensors configured to provide a reference sensor signal. For example, a reference sensor may be an optical sensor in the optical array (e.g., array of same type of sensors or different types of sensors), or may be a non-optical sensor (e.g., thermocouple, force transducer, piezoelectric element). The reference sensor may help differentiate between different physical signals. For example, the reference sensor may have a predetermined or known sensitivity to a particular physical signal of interest (e.g., pressure, temperature, acoustic wave, a combination thereof, and/or the like). The reference sensor may therefore be used to calibrate one or more optical sensors in the optical array, and/or verify the sensitivity of the other optical sensors in the optical array. For example, if the reference sensor is more sensitive to temperature for a predetermined range of frequencies, the output of the reference sensor may be analyzed to evaluate sensor responses within that predetermined range of frequencies. A sensor that may be similar to the reference sensor may be calibrated based on the sensor responses of the reference sensor.

In some variations, using two or more sensors may help differentiate between two physical signals. For example, an optical array with a first optical sensor may be configured to provide a first sensor signal (and first plurality of sensor responses) and a second optical sensor may be configured to provide a second sensor signal (and second plurality of sensor responses), wherein some variations, the first and second optical sensors may have different sensitivities to different physical signals. Using such an optical array, a method of multi-dimensional sensing may include generating a first measurement signal indicative of a first physical signal (e.g., target physical signal), wherein the first measurement signal is based on (i) the first plurality of sensor responses for the first optical sensor, (ii) the second plurality of sensor responses for the second optical sensor, and (iii) the sensitivities of the first and second optical sensors to the first physical signal and a second physical signal. Furthermore, the method may include generating a second measurement signal indicative of the second physical signal using (i), (ii), and (iii).

As an illustrative example, consider a first optical sensor that may respond to both pressure and temperature signals. The first sensor's sensor signal may change by one unit in response to a change of one unit temperature (temperature sensitivity S_{1T}=1) and also may change by one unit in response to a change of one unit pressure (pressure sensitivity S_{1P}=1). In such a scenario, if the first sensor's sensor signal changes by two units, it may be difficult to identify if the change was caused entirely by a two unit change in temperature, a two unit change in pressure, or a one unit change each in temperature and pressure. However, using a second sensor that has twice as strong a response to temperature as the first sensor (temperature sensitivity S_{2T}=2) but the same response to pressure as the first sensor (pressure sensitivity S_{2P}=1), in conjunction with the first sensor, may deconvolve the temperature and pressure. This is further illustrated in the table below.

| | Temperature | Pressure |
|---|---|---|
| Sensitivity of first sensor | S_{1T} = 1 | S_{1P} = 1 |
| Sensitivity of second sensor | S_{2T} = 2 | S_{2P} = 1 |
| Change in temperature (ΔT) and | ΔT = 1, ΔP = 1 | |
| pressure (ΔP) | | |
| Output of first sensor (O₁₎ | 1 + 1 = 2 | |
| Output of second sensor (O₂₎ | 2 + 1 = 3 | |

As seen in the table above, in response to a temperature change of one unit and a pressure change of one unit, the total output of the first sensor is different from the total output of the second sensor. The change in temperature may be determined, for example, by the below sequence of equations: First, the outputs of the first sensor and second sensor may be expressed as Equations (7) and (8), respectively:

$${O}_{1} = {S}_{1 T} * Δ T + {S}_{1 P} * Δ P \qquad ( 7 )$$

$${O}_{2} = {S}_{2 T} * Δ T + {S}_{2 P} * Δ P \qquad ( 8 )$$

### From Equations (7) and (8):

$$\begin{matrix}Δ P = ( {O}_{1} - {S}_{1 T} * Δ T ) / {S}_{1 P} & ( 9 ) \\ Δ P = ( {O}_{2} - {S}_{2 T} * Δ T ) / {S}_{2 P} & ( 10 )\end{matrix}$$

By equating Equations (9) and (10), change in temperature may be derived as follows:

$$( {O}_{2} - {S}_{2 T} * Δ T ) / {S}_{2 P} = ( {O}_{1} - {S}_{1 T} * Δ T ) / {S}_{1 P} \qquad ( 11 )$$

$${S}_{1 P} ( {O}_{2} - {S}_{2 T} * Δ T ) = {S}_{2 P} ( {O}_{1} - {S}_{1 T} * Δ T ) \qquad ( 12 )$$

$${S}_{1 P} {O}_{2} - {S}_{2 T} {S}_{2 T} * Δ T = {S}_{2 P} {O}_{1} - {S}_{2 P} {S}_{1 T} * Δ T \qquad ( 13 )$$

$${S}_{1 P} {O}_{2} - {S}_{2 P} {O}_{1} = {S}_{1 P} {S}_{2 T} * Δ T - {S}_{2 P} {S}_{1 T} * Δ T = ( {S}_{1 P} {S}_{2 T} - {S}_{2 P} {S}_{1 T} ) * Δ T \qquad ( 14 )$$

$$Δ T = ( {S}_{1 P} {O}_{2} - {S}_{2 P} {O}_{1} ) / ( {S}_{1 P} {S}_{2 T} - {S}_{2 P} {S}_{1 T} ) \qquad ( 15 )$$

Similarly, change in pressure may be determined by the below sequence of equations. From Equations (7) and (8) above:

$$Δ T = ( {O}_{1} - {S}_{1 P} * Δ P ) / {S}_{1 T} \qquad ( 16 )$$

$$Δ T = ( {O}_{2} - {S}_{2 P} * Δ P ) / {S}_{2 T} \qquad ( 17 )$$

By equating Equations (15) and (16), change in pressure may be derived as follows:

$$( {O}_{2} - {S}_{2 P} * Δ P ) / {S}_{2 T} = ( {O}_{1} - {S}_{1 P} * Δ P ) / {S}_{1 T} \qquad ( 18 )$$

$${S}_{1 T} ( {O}_{2} - {S}_{2 P} * Δ P ) = {S}_{2 T} ( {O}_{1} - {S}_{1 P} * Δ P ) \qquad ( 19 )$$

$${S}_{1 T} {O}_{2} - {S}_{1 T} {S}_{2 P} * Δ P = {S}_{2 T} {O}_{1} - {S}_{2 T} {S}_{1 P} * Δ P \qquad ( 20 )$$

$${S}_{1 T} {O}_{2} - {S}_{2 T} {O}_{1} = {S}_{1 T} {S}_{2 P} * Δ P - {S}_{2 T} {S}_{1 P} * Δ P = ( {S}_{1 T} {S}_{2 P} - {S}_{2 T} {S}_{1 P} ) * Δ P \qquad ( 21 )$$

$$Δ P = ( {S}_{1 T} {O}_{2} - {S}_{2 T} {O}_{1} ) / ( {S}_{1 T} {S}_{2 P} - {S}_{2 T} {S}_{1 P} ) \qquad ( 22 )$$

Equations (15) and (22) are solvable when the first and second optical sensors have different sensitivities such that the denominator of Equations (15) and (22) is non-zero (S_{1T} S_{2P}≠S_{2T} S_{1P}). In other words, the differential sensing using multiple sensors with different sensitivities may be possible when, for example, (i) the product of the sensitivity of the first sensor to the first physical signal and the sensitivity of the second sensor to the second physical signal and (ii) the product of the sensitivity of the second sensor to the first physical signal and the sensitivity of the second sensor to the first physical signal are such that (i) and (ii) are not equal to each other. Thus, where Equation (15) is solvable, the measurement signal for ΔT can be generated from the sensor responses, and where Equation (22) is solvable, the measurement signal for ΔP can be generated from the sensor responses. An analogous approach may be used to differentiate between other physical signals as well.

In the above equations, S_{1T} represents the sensitivity of the first sensor to temperature, S_{1P} represents the sensitivity of the first sensor to pressure, S_{2P} represents the sensitivity of the second sensor to pressure, S_{2T} represents the sensitivity of the second sensor to temperature, O₁ represents the output of the first sensor, and O₂ represents the output of the second sensor. ΔT represents the change in temperature. ΔP, which represents the change in pressure may be computed in a manner similar to ΔT.

In this manner, the systems and methods described herein enable accurate measurement of multiple physical signals using a single optical sensor. The technology described herein may be used, for example, in devices during surgery or other medical procedures. For instance, the technology described herein may be used in applications that require temperature and pressure changes to be detected simultaneously, such as monitoring multiple vital signs in a patient in real time during a heart surgery to ensure patient safety.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for multi-dimensional sensing, the method comprising:
receiving a sensor signal from a single optical sensor proximate to a measurement region;
determining a plurality of sensor responses from the sensor signal; and
generating a plurality of measurement signals from the plurality of sensor responses, wherein each of the measurement signals corresponds to a different respective physical signal of the measurement region.

2. The method of claim 1, wherein a sensor response of the plurality of sensor responses is selected from the group consisting of one or more of a mode shift, a baseline drift, a mode split, a mode broadening, and any combination thereof.

3. The method of claim 2, wherein the sensor response comprises a mode shift comprising one or more of a change in a resonant frequency, a change in depth, and a change in shape.

4. The method of claim 1, wherein the physical signals of an environment comprises two or more of a temperature, a pressure, and an acoustic wave of the environment.

5. The method of claim 1, wherein generating the plurality of measurement signals further comprises decoupling at least a portion of the physical signals.

6. The method of claim 5, wherein decoupling at least a portion of the physical signals further comprises:
analyzing a first sensor response of the plurality of sensor responses for a first time period;
generating a first measurement signal for the first time period based on the first sensor response;
analyzing a second sensor response of the plurality of sensor responses for a second time period; and
generating a second measurement signal for the second time period based on the second sensor response.

7. The method of claim 6, further comprising:
analyzing a third sensor response of the plurality of sensor responses for a third time period; and
generating a third measurement signal for the third time period based on the third sensor response.

8. The method of claim 6, wherein the first sensor response comprises a mode shift and the first measurement signal corresponds to temperature, and wherein the second sensor response comprises a baseline shift and the second measurement signal corresponds to pressure.

9. The method of claim 5, wherein decoupling at least a portion of the physical signals comprises selectively altering the environment based on a targeted physical signal.

10. The method of claim 9, wherein decoupling at least a portion of the physical signals comprises suppressing one or more of the physical signals different from the targeted physical signal.

11. The method of claim 10, wherein suppressing one or more of the physical signals comprises adjusting the environment such that the one or more physical signals different from the target physical signal is within a first sensitivity signal region of the optical sensor.

12. The method of claim 10, wherein suppressing the one or more physical signals comprises modifying one or more of a temperature, a pressure, and an acoustic property of the environment.

13. The method of claim 9, wherein decoupling at least a portion of the physical signals comprises increasing a sensitivity of the optical sensor to the targeted physical signal.

14. The method of claim 13, wherein increasing the sensitivity of the optical sensor comprises adjusting an environment of the optical sensor such that the target physical signal is within a second sensitivity signal region of the optical sensor different from the first sensitivity signal region.

15. The method of claim 14, wherein increasing the sensitivity comprises:
analyzing a first sensor response of the plurality of sensor responses for a first time period associated with the first sensitivity signal region; and
generating a first measurement signal corresponding to the target physical signal.

16. The method of claim 1, wherein generating the plurality of measurement signals comprises associating a first sensor response of the plurality of sensor responses with a first physical signal, and associating a second sensor response of the plurality of sensor responses with a second physical signal.

17. The method of claim 1, wherein generating the plurality of measurement signals comprises applying a signal transformation function to the plurality of sensor responses.

18. The method of claim 17, wherein the signal transformation function comprises a signal transformation matrix.

19. The method of claim 1, wherein the optical sensor comprises a first optical sensor of an array of optical sensors.

20. The method of claim 1, wherein the sensor signal comprises a first sensor signal and the plurality of sensor responses comprises a first plurality of sensor responses, wherein the method further comprises:
receiving a second sensor signal from a second optical sensor of the array of optical sensors;
determining a second plurality of sensor responses from the second optical sensor; and
generating a first measurement signal indicative of a first physical signal, wherein the first measurement signal is based on the first plurality of sensor responses for the first optical sensor, the second plurality of sensor responses for the second optical sensor, and sensitivities of the first and second optical sensors to the first physical signal and a second physical signal.

21. The method of claim 1, wherein the plurality of measurement signals is generated at least in part based on a reference signal from a reference sensor.

22. The method of claim 1, wherein the optical sensor comprises an interference-based optical sensor.

23. The method of claim 22, wherein the optical sensor comprises an optical resonator or an optical interferometer.

24. The method of claim 23, wherein the optical sensor comprises a whispering gallery mode (WGM) resonator.

25. The method of claim 23, wherein the optical sensor comprises one or more of a microbubble optical resonator, a microsphere resonator, a micro-toroid resonator, a micro-ring resonator, and a micro-disk optical resonator.

26. A system for multi-dimensional sensing of a measurement region, the system comprising:
an optical sensor;
a signal processor configured to:
receive a sensor signal from the optical sensor;
determine a plurality of sensor responses from the sensor signal; and
generate a plurality of measurement signals from the plurality of sensor responses, wherein each measurement signal corresponds to a different respective physical signal of the measurement region.

27. The system of claim 26, wherein a sensor response of the plurality of sensor responses is selected from the group consisting of one or more of a mode shift, a baseline drift, a mode split, and a mode broadening.

28. The system of claim 27, wherein the sensor response comprises a mode shift comprising one or more of a change in resonant frequency, a change in depth, and a change in shape.

29. The system of claim 26, wherein the physical signals of an environment comprises two or more of a temperature, a pressure, and an acoustic wave of the environment.

30. The system of claim 26, wherein the optical sensor comprises an interference-based optical sensor.

31. The system of claim 30, wherein the optical sensor comprises an optical resonator or an optical interferometer.

32. The system of claim 31, wherein the optical sensor comprises a whispering gallery mode (WGM) resonator.

33. The system of claim 31, wherein the optical sensor comprises one or more of a microbubble resonator, a microsphere resonator, a microtoroid resonator, a microring resonator, microbottle resonator, microcylinder, and a microdisk optical resonator.

34. The system of claim 33, wherein the optical sensor comprises a microring resonator comprising one or more of a cross-sectional shape of a circle, a racetrack, and an ellipse.

35. The system of claim 26, wherein the optical sensor comprises a first optical sensor of an array of optical sensors and wherein the system further comprises the array of optical sensors.

36. The system of claim 35, wherein the array of optical sensors comprises a second optical sensor, and wherein the first optical sensor has a higher sensitivity to a first physical signal than the second optical sensor and the second optical sensor has a higher sensitivity to a second physical signal than the first optical sensor, the first physical signal different from the second physical signal.

37. The system of claim 36, wherein an environment of the first optical sensor is configured to enhance the first physical signal or an environment of the second optical sensor is configured to suppress the first physical signal.

38. The system of claim 36, wherein an environment of the first optical sensor is configured to suppress the second physical signal or an environment of the second optical sensor is configured to enhance the second physical signal.

39. The system of claim 26, further comprising a reference sensor configured to provide a reference signal corresponding to one or more of the physical signals of the measurement region.

40. The system of claim 39, wherein the reference sensor comprises an optical sensor.

41. The system of claim 39, wherein the reference sensor comprises a non-optical sensor.
